SEQUENCE LISTING (48 sequences)

SEQ ID NO: 1 - protein, 2511 aa, Homo sapiens
MEEVVIAGMSGKLPESENLQEFWDNLIGGVDMVTDDDRRWKAGLYGLPRRSGKLKDLSRFDASFFGVHPKQAHTMDPQLRLLLEVTYEAIVDGGINPDSLRGTHTGVWVGVSGSETSEALSRDPETLVGYSMVGCQRAMMANRLSFFFDFRGPSIALDTACSSSLMALQNAYQAIHSGQCPAAIVGGINVLLKPNTSVQFLRLGMLSPEGTCKAFDTAGNGYCRSEGVVAVLLTKKSLARRVYATILNAGTNTDGFKEQGVTFPSGDIQEQLIRSLYQSAGVAPESFEYIEAHGTGTKVGDPQELNGITRALCATRQEPLLIGSTKSNMGHPEPASGLAALAKVLLSLEHGLWAPNLHFHSPNPEIPALLDGRLQVVDQPLPVRGGNVGINSFGFGGSNVHIILRPNTQPPPAPAPHATLPRLLRASGRTPEAVQKLLEQGLRHSQDLAFLSMLNDIAAVPATAMPFRGYAVLGGERGGPEVQQVPAGERPLWFICSGMGTQWRGMGLSLMRLDRFRDSILRSDEAVKPFGLKVSQLLLSTDESTFDDIVHSFVSLTAIQIGLIDLLSCMGLRPDGIVGHSLGEVACGYADGCLSQEEAVLAAYWRGQCIKEAHLPPGAMAAVGLSWEECKQRCPPGVVPACHNSKDTVTISGPQAPVFEFVEQLRKEGVFAKEVRTGGMAFHSYFMEAIAPPLLQELKKVIREPKPRSARWLSTSIPEAQWHSSLARTSSAEYNVNNLVSPVLFQEALWHVPEHAVVLEIAPHALLQAVLKRGLKPSCTIIPLMKKDHRDNLEFFLAGIGRLHLSGIDANPNALFPPVEFPAPRGTPLISPLIKWDHSLAWDVPAAEDFPNGSGSPSAAIYNIDTSSESPDHYLVDHTLDGRVLFPATGYLSIVWKTLARALGLGVEQLPVVFEDVVLHQATILPKTGTVSLEVRLLEASRAFEVSENGNLVVSGKVYQWDDPDPRLFDHPESPTPNPTEPLFLAQAEVYKELRLRGYDYGPHFQGILEASLEGDSGRLLWKDNWVSFMDTMLQMSILGSAKHGLYLPTRVTAIHIDPATHRQKLYTLQDKAQVADVVVSRWLRVTVAGGVHISGLHTESAPRRQQEQQVPILEKFCFTPHTEEGCLSERAALQEELQLCKGLVQALQTKVTQQGLKMVVPGLDGAQIPRDPSQQELPRLLSAACRLQLNGNLQLELAQVLAQERPKLPEDPLLSGLLDSPALKACLDTAVENMPSLKMKVVEVLAGHGHLYSRIPGLLSPHPLLQLSYTATDRHPQALEAAQAELQQHDVAQGQWDPADPAPSALGSADLLVCNCAVAALGDPASALSNMVAALREGGFLLLHTLLRGHPLGDIVAFLTSTEPQYGQGILSQDAWESLFSRVSLRLVGLKKSFYGSTLFLCRRPTPQDSPIFLPVDDTSFRWVESLKGILADEDSSRPVWLKAINCATSGVVGLVNCLRREPGGNRLRCVLLSNLSSTSHVPEVDPGSAELQKVLQGDLVMNVYRDGAWGAFRHFLLEEDKPEEPTAHAFVSTLTRGDLSSIRWVCSSLRHAQPTCPGAQLCTVYYASLNFRDIMLATGKLSPDAIPGKWTSQDSLLGMEFSGRDASGKRVMGLVPAKGLATSVLLSPDFLWDVPSNWTLEEAASVPVVYSTAYYALVVRGRVRPGETLLIHSGSGGVGQAAIAIALSLGCRVFTTVGSAEKRAYLQARFPQLDSTSFANSRDTSFEQHVLWHTGGKGVDLVLNSLAEEKLQASVRCLATHGRFLEIGKFDLSQNHPLGMAIFLKNVTFHGVLLDAFFNESSADWREVWALVQAGIRDGVVRPLKCTVFHGAQVEDAFRYMAQGKHIGKVVVQVLAEEPEAVLKGAKPKLMSAISKTFCPAHKSYIIAGGLGGFGLELAQWLIQRGVQKLVLTSRSGIRTGYQAKQVRRWRRQGVQVQVSTSNISSLEGARGLIAEAAQLGPVGGVFNLAVVLRDGLLENQTPEFFQDVCKPKYSGTLNLDRVTREACPELDYFVVFSSVSCGRGNAGQSNYGFANSAMERICEKRRHEGLPGLAVQWGAIGDVGILVETMSTNDTIVSGTLPQRMASCLEVLDLFLNQPHMVLSSFVLAEKAAAYRDRDSQRDLVEAVAHILGIRDLAAVNLDSSLADLGLDSLMSVEVRQTLERELNLVLSVREVRQLTLRKLQELSSKADEASELACPTPKEDGLAQQQTQLNLRSLLVNPEGPTLMRLNSVQSSERPLFLVHPIEGSTTVFHSLASRLSIPTYGLQCTRAAPLDSIHSLAAYYIDCIRQVQPEGPYRVAGYSYGACVAFEMCSQLQAQQSPAPTHNSLFLFDGSPTYVLAYTQSYRAKLTPGCEAEAETEAICFFVQQFTDMEHNRVLEALLPLKGLEERVAAAVDLIIKSHQGLDRQELSFAARSFYYKLRAAEQYTPKAKYHGNVMLLRAKTGGAYGEDLGADYNLSQVCDGKVSVHVIEGDHRTLLEGSGLESIISIIHSSLAEPRVSVREG

SEQ ID NO: 2 - protein, 605 aa, Homo sapiens
MSQLSSTLKRYTESARYTDAHYAKSGYGAYTPSSYGANLAASLLEKEKLGFKPVPTSSFLTRPRTYGPSSLLDYDRGRPLLRPDITGGGKRAESQTRGTERPLGSGLSGGSGFPYGVTNNCLSYLPINAYDQGVTLTQKLDSQSDLARDFSSLRTSDSYRIDPRNLGRSPMLARTRKELCTLQGLYQTASCPEYLVDYLENYGRKGSASQVPSQAPPSRVPEIISPTYRPIGRYTLWETGKGQAPGPSRSSSPGRDGMNSKSAQGLAGLRNLGNTCFMNSILQCLSNTRELRDYCLQRLYMRDLHHGSNAHTALVEEFAKLIQTIWTSSPNDVVSPSEFKTQIQRYAPRFVGYNQQDAQEFLRFLLDGLHNEVNRVTLRPKSNPENLDHLPDDEKGRQMWRKYLEREDSRIGDLFVGQLKSSLTCTDCGYCSTVFDPFWDLSLPIAKRGYPEVTLMDCMRLFTKEDVLDGDEKPTCCRCRGRKRCIKKFSIQRFPKILVLHLKRFSESRIRTSKLTTFVNFPLRDLDLREFASENTNHAVYNLYAVSNHSGTTMGGHYTAYCRSPGTGEWHTFNDSSVTPMSSSQVRTSDAYLLFYELASPPSRM

SEQ ID NO: 3 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 4 - protein, 382 aa, Homo sapiens
MALQGISVVELSGLAPGPFCAMVLADFGARVVRVDRPGSRYDVSRLGRGKRSLVLDLKQPRGAAVLRRLCKRSDVLLEPFRRGVMEKLQLGPEILQRENPRLIYARLSGFGQSGSFCRLAGHDINYLALSGVLSKIGRSGENPYAPLNLLADFAGGGLMCALGIIMALFDRTRTGKGQVIDANMVEGTAYLSSFLWKTQKLSLWEAPRGQNMLDGGAPFYTTYRTADGEFMAVGAIEPQFYELLIKGLGLKSDELPNQMSMDDWPEMKKKFADVFAEKTKAEWCQIFDGTDACVTPVLTFEEVVHHDHNKERGSFITSEEQDVSPRPAPLLLNTPAIPSFKRDPFIGEHTEEILEEFGFSREEIYQLNSDKIIESNKVKASL

SEQ ID NO: 5 - protein, 480 aa, Homo sapiens [sequence omitted]

SEQ ID NO: 6 - protein, 8 aa, Homo sapiens
AAEQYTPK

SEQ ID NO: 7 - protein, 10 aa, Homo sapiens
VFTTVGSAEK

SEQ ID NO: 8 - protein, 11 aa, Homo sapiens
AFDTAGNGYCR

SEQ ID NO: 9 - protein, 13 aa, Homo sapiens
ADEASELACPTPK

SEQ ID NO: 10 - protein, 9 aa, Homo sapiens
GYAVLGGER

SEQ ID NO: 11 - protein, 9 aa, Homo sapiens
YSGTLNLDR

SEQ ID NO: 12 - protein, 8 aa, Homo sapiens
QELSFAAR

SEQ ID NO: 13 - protein, 11 aa, Homo sapiens
LQVVDQPLPVR

SEQ ID NO: 14 - protein, 11 aa, Homo sapiens
FDASFFGVHPK

SEQ ID NO: 15 - protein, 10 aa, Homo sapiens
GTPLISPLIK

SEQ ID NO: 16 - protein, 11 aa, Homo sapiens
SEGVVAVLLTK

SEQ ID NO: 17 - protein, 9 aa, Homo sapiens
VLEALLPLK

SEQ ID NO: 18 - protein, 15 aa, Homo sapiens
VVVQVLAEEPEAVLK

SEQ ID NO: 19 - protein, 15 aa, Homo sapiens
AFEVSENGNLVVSGK

SEQ ID NO: 20 - protein, 13 aa, Homo sapiens
SLLVNPEGPTLMR

SEQ ID NO: 21 - protein, 13 aa, Homo sapiens
GVDLVLNSLAEEK

SEQ ID NO: 22 - protein, 11 aa, Homo sapiens
SFYGSTLFLCR

SEQ ID NO: 23 - protein, 11 aa, Homo sapiens
AALQEELQLCK

SEQ ID NO: 24 - protein, 12 aa, Homo sapiens
VGDPQELNGITR

SEQ ID NO: 25 - protein, 10 aa, Homo sapiens
VYQWDDPDPR

SEQ ID NO: 26 - protein, 9 aa, Homo sapiens
TGTVSLEVR

SEQ ID NO: 27 - protein, 9 aa, Homo sapiens
LDSQSDLAR

SEQ ID NO: 28 - protein, 12 aa, Homo sapiens
FLLDGLHNEVNR

SEQ ID NO: 29 - protein, 12 aa, Homo sapiens
TYGPSSLLDYDR

SEQ ID NO: 30 - protein, 10 aa, Homo sapiens
IGDLFVGQLK

SEQ ID NO: 31 - protein, 10 aa, Homo sapiens
LTTFVNFPLR

SEQ ID NO: 32 - protein, 6 aa, Homo sapiens
YYSALR

SEQ ID NO: 33 - protein, 8 aa, Homo sapiens
ESTENVPR

SEQ ID NO: 34 - protein, 10 aa, Homo sapiens
EEIYQLNSDK

SEQ ID NO: 35 - protein, 8 aa, Homo sapiens
SSLWEAPR

SEQ ID NO: 36 - protein, 12 aa, Homo sapiens
LSGFGQSGSFCR

SEQ ID NO: 37 - protein, 7 aa, Homo sapiens
FADVFAK

SEQ ID NO: 38 - protein, 16 aa, Homo sapiens
EEIYQLNSDKIIESNK

SEQ ID NO: 39 - protein, 7 aa, Homo sapiens
SLVLDLK

SEQ ID NO: 40 - protein, 10 aa, Homo sapiens
RSDVLLEPFR

SEQ ID NO: 41 - protein, 10 aa, Homo sapiens
LQLGPEILQR

SEQ ID NO: 42 - protein, 9 aa, Homo sapiens
SDVLLEPFR

SEQ ID NO: 43 - protein, 17 aa, Homo sapiens
LAGHDINYLALSGVLSK

SEQ ID NO: 44 - DNA, 8481 bp, Homo sapiens
gagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctccaaaaaaaaaaaaaaaaaaaaaaaa

SEQ ID NO: 45 - DNA, 3748 bp, Homo sapiens
agtgacgcgagacgcggggtgtggctctgccggcccaggcgcgatgaggcggctgcccgctgggtggcgccgatttcccggggaggtcccttctgggcccccggcggaggtgggagagagtcaggcaggagccgaggccggggagccctcttcgtcagctggtgctcactgcgccgcgccagcgccagccgggactcacccgcagctccatgcttgtgcccggttcgactcgtccatactccaagaagaggcagcccatgaggctcccagtccccactgagtgccaccctgaaggatgtcccagctctcctccaccctgaagcgctacacagaatcggcccgctacacagatgcccactatgccaagtcgggctatggtgcctacaccccgtcctcctatggggccaatctggctgcctccttactggagaaggagaaacttggtttcaagccggtccccaccagcagcttcctcacccgtccccgtacctatggcccctcctccctcctggactatgaccggggccgccccctgctgagacccgacatcactgggggtggtaagcgggcagagagccagacccggggtactgagcggcctttaggcagtggcctcagcgggggcagcggattcccttatggagtgaccaacaactgcctcagctacctgcccatcaatgcctatgaccagggggtgaccctaacccagaagctggacagccaatcagacctggcccgggatttctccagcctccggacctcagatagctaccggatagaccccaggaacctgggccgcagccccatgctggcccggacgcgcaaggagctctgcaccctgcaggggctctaccagacagccagctgccctgaatacctggtcgactacctggagaactatggtcgcaagggcagtgcatctcaggtgccctcccaggcccctccctcacgagtccctgaaatcatcagcccaacctaccgacccattggccgctacacgctgtgggagacgggaaagggtcaggcccctgggcccagccgctccagctccccgggaagagacggcatgaattctaagagtgcccagggtctggctggtcttcgaaaccttgggaacacgtgcttcatgaactcaattctgcagtgcctgagcaacactcgggagttgagagattactgcctccagaggctctacatgcgggacctgcaccacggcagcaatgcacacacagccctcgtggaagagtttgcaaaactaattcagaccatatggacttcatcccccaatgatgtggtgagcccatctgagttcaagacccagatccagagatacgcaccgcgctttgttggctataatcagcaggatgctcaggagttccttcgctttcttctggatgggctccataacgaggtgaaccgagtgacactgagacctaagtccaaccctgagaacctcgatcatcttcctgatgacgagaaaggccgacagatgtggagaaaatatctagaacgggaagacagtaggatcggggatctctttgttgggcagctaaagagctcgctgacgtgtacagattgtggttactgttctacggtcttcgaccccttctgggacctctcactgcccattgctaagcgaggttatcctgaggtgacattaatggactgcatgaggctcttcaccaaagaggatgtgcttgatggagatgaaaagccaacatgctgtcgctgccgaggcagaaaacggtgtataaagaagttctccatccagaggttcccaaagatcttggtgctccatctgaagcggttctcagaatccaggatccgaaccagcaagctcacaacatttgtgaacttccccctaagagacctggacttaagagaatttgcctcagaaaacaccaaccatgctgtttacaacctgtacgctgtgtccaatcactccggaaccaccatgggtggccactatacagcctactgtcgcagtccagggacaggagaatggcacactttcaacgactccagcgtcactcccatgtcctccagccaagtgcgcaccagcgacgcctacctgctcttctacgaactggccagcccgccctcccgaatgtagcgccaggagccacgtcccttctcccttccccgtggtggccccgctccctaaattttttaaaaagacaaaaacaaaacaacaacaacaacacacaaacctgacaagagaaaaacaaacctgaagctgccgagcaggagtggatgcagcctgatcagggtctggagcaaggagccgggctttcctgagctgtggcccggcagggaagatcgcctggacgtggagccagcatcgccccgtgccctcggcgtttgcatttgtaaacttgtggtcttcctatgtgtcagaaacaactgtgtcttgggggggaagaccctcgctgcgccgcttcccgccgcagcgcccgcgcctccgaggggacagcgccctctggagctcgctgggagcatcaccgcctggacgcccgcgccgcggaggagccggcgcccatctccacccgcacggctcgccggtccagagccatgagccaagagccctcttcacgctgctaactccaggggacagacgaagggacatctttggaaaacgctggttttggtttttaaaaagcccaactttttttttttaatttccataactaaagtgttcagactggagtgctctccttcaggcctcttcatagctgggacgttgcactggtccttttattgcttttccaagtacaactttctaatgctagccctccgtggtgctaggtgggcgttggccaggccccaagcacagccacagtagacctgggatctaaaacaagtttctgttttgggggtttgggttttttttttttttttttaatgttttgaatggaatttagttgcctccaagaattgtgccttatagcatttggggaccagggggtaactgcccctcctgaaatatccctcagcctcttcccttttccccagtgctctgttcaaacccgcctgggaaagggatcctgcccttagccctggctcgttgtgcattgcagtgaggcaaagaagaaagcaggtagattccttccgacagggcatcaagttcttcccgcccacgtcctctagcccacccctggtctgctccccagctgtttgaaggatagcacaagcccctcgtccctagagcttctctcccttttatttattctcttaacatccctttccccctggccttcctgcccccgcccccttctcagagcctcctagacaataggccctttggaccgagtttctcagggatgcccaggccacccctcagctcttcttagcgctggtctccagtcctgccctgggagctggagcctgggtatttggggacatcttgcctcagttgtatggttctttcctgtgggctcaattttgccctacatagttggataaaactctgtgctgtcctggagagtaaagctgttcacccacacagctgggcccggcttgtgccccgtggagcctggcacattccaggctcctaggaggaggcatcagagaaagacaccctgagttttactggcctgacacccttctccagagaagacctgtgaacctgagcccaagggcaagtgtacacttgtttactgtgtaagcaagagtagaagaatgtctaatgtacagtggaaccttgtacagaataaataatagctttgagaaatcaaaaaaaaaaaaaaaa

SEQ ID NO: 46 - DNA, 576 bp, Homo sapiens
gcaccccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattcaaaagtgaaaaaaaaaaaaaaaa

SEQ ID NO: 47 - DNA, 3352 bp, Homo sapiens
ggggcgtggcgccggggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaa

SEQ ID NO: 48 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

(19) United States
(12) Patent Application Publication
(10) Pub. No.: US 2015/0133342 A1
(43) Pub. Date: May 14, 2015

(54) MRM-MS SIGNATURE ASSAY

(71) Applicant: Nuclea Biotechnologies, Inc., Pittsfield, MA (US)

(72) Inventors: Sun W. Tam, Westborough, MA (US); Ying Xu, Newton, MA (US); Patrick J. Muraca, Pittsfield, MA (US)

(21) Appl. No.: 14/398,794

(22) PCT Filed: May 3, 2013
(86) PCT No.: PCT/US13/39356

§ 371 Date: Nov. 4, 2014

Related U.S. Application Data
(60) Provisional application No. 61/697,343, filed on Sep. 6, 2012.
(60) Provisional application No. 61/670,778, filed on Jul. 12, 2012.
(60) Provisional application No. 61/642,596, filed on May 4, 2012.

(51) Int. Cl. G01N 33/68; C12Q 1/37

(52) U.S. Cl. CPC G01N 33/6848; C12Q 1/37; G01N 2333/5755; G01N 2333/99; G01N 2560/00; G01N 2800/7028
USPC 506/12; 435/23; 506/18; 530/328; 530/327; 530/326

(57) ABSTRACT

The present invention relates to mass spectrometry methods employing multiple reaction monitoring (MRM) in the field of cancer therapeutics, specifically prostate cancer and melanoma.

### RELATED APPLICATIONS

The present application claims the benefits of U.S. Provisional Application Ser. No. 61/642,596, filed May 4, 2012, U.S. Provisional Application Ser. No. 61/670,778, filed Jul. 12, 2012, U.S. Provisional Application Ser. No. 61/697,343, filed Sep. 6, 2012, the contents of which by references are incorporated herein in their entireties.

### REFERENCE TO SEQUENCE LISTING

The present application is being filed along with a Sequence Listing in electronic format. The Sequence Listing is provided as a file entitled SEQLST20151016PCT.txt, created on May 2, 2013, which is 67,673 bytes in size. The information in the electronic format of the sequence listing is incorporated herein by reference in its entirety.

### FIELD OF THE INVENTION

The present invention relates to the development of protein signature assays by multiple reaction monitoring mass spectroscopy (MRM-MS).

### BACKGROUND OF THE INVENTION

Prostate cancer is the most commonly diagnosed malignancy in men and the second leading cause of cancer related death in USA. Recently, clinical trials have focused on targeting the metabolic pathways in cancer namely the lipogenic related molecular targets (Zadra G et al., New Strategies in Prostate Cancer: Targeting Lipogenic Pathways and the Energy Sensor AMPK. (2010), Clin Cancer Res; 16(13): 3322-3328.) As such, fatty acid synthase (FASN, OA-519) and its affiliated molecular partners, such as USP2a (Priolo C et al., The Isopeptidase USP2a Protects Human Prostate Cancer from Apoptosis. (2006) Cancer Research 66:8625-8632.), Neuropeptide Y (NPY) (Nagakawa O et al., Effect of prostatic neuropeptides on migration of prostate cancer cell lines. (2001), Int J Urol. 8(2): 65-70.), AMACR (Rubin M A et al., Decreased alpha-methylacyl CoA racemase expression in localized prostate cancer is associated with an increased rate of biochemical recurrence and cancer-specific death. (2005) Cancer Epidemiol Biomarkers Prev.; 14(6): 1424-32.) and pAKT (Van de Sande T, et al., High-level expression of fatty acid synthase in human prostate cancer tissues is linked to activation and nuclear localization of Akt/PKB, (2005) J Pathol, 206(2):214-219.) have become very attractive diagnostic and therapeutic targets in this pathway for discovery.

The levels of FASN as well as any affiliated molecular targets therefore represent valuable biomarkers, alone or in combination, in the identification of patient populations that would benefit most from a FASN directed treatment in prostate cancer. In conjunction with the interest in lipogenic pathways, the U.S. Preventive Services Task Force (USPSTF) has retreated from the recommendation to use prostate-specific antigen (PSA)-based screening for prostate cancer. The task force also rejects the PSA test for surveillance after diagnosis and/or treatment of prostate cancer. The changes in diagnostic and clinical trends have created a new urgency for novel biomarkers that can replace PSA in prostate cancer diagnosis and treatment.

To date, there is no FDA approved FASN, USP2a, NPY, AMACR or pAKT ELISA assay or other quantitative assay to meet this long felt need. The discovery, verification and validation of disease specific protein biomarkers are best tested in biofluids with a high throughput and sensitive bioassay. ELISA assays can normally meet these criteria. However, the shortcomings of the ELISA include high development costs, long lead times for assay development, and the inability to multiplex or to measure protein modifications. For instance, specific phosphorylated forms of FASN or specific modified forms of USP2a might have better predictive value in certain stages of cancer progression.

Mass spectrometry is performed using a mass spectrometer which includes an ion source for ionizing the fractionated sample and creating charged molecules for further analysis. For example ionization of the sample may be performed by electrospray ionization (ESI), atmospheric pressure chemical ionization (APCI), photoionization, electron ionization, fast atom bombardment (FAB)/liquid secondary ionization (LSIMS), matrix assisted laser desorption ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

After the sample has been ionized, the positively charged or negatively charged ions thereby created may be analyzed to determine a mass-to-charge ratio (i.e., m/z). Suitable analyzers for determining mass-to-charge ratios include quadropole analyzers, ion traps analyzers, and time-of-flight analyzers. The ions may be detected using several detection modes. For example, selected ions may be detected (i.e., using a selective ion monitoring mode (SIM)), or alternatively, ions may be detected using a scanning mode, e.g., multiple reaction monitoring (MRM) or selected reaction monitoring (SRM).

The present invention relates to mass spectrometry methods employing multiple reaction monitoring (MRM) in the field of cancer diagnosis and therapeutics, specifically prostate cancer.

### SUMMARY OF THE INVENTION

The present invention provides methods, kits and peptide compositions for determining the presence and concentration of biomarkers and/or their affiliated molecular partners using the multiple reaction monitoring (MRM)-mass spectrometry (MS) based assay platform. In one aspect, the present invention relates to MRM-MS based assays to overcome the development issues associated with high development costs, long lead times for assay development and the inability to multiplex or to measure protein modifications of the assays currently used in the art.

According to the present invention, described herein is a method of determining the concentration of a prostate cancer biomarker and/or affiliated molecular partner in a sample. As used herein a “prostate cancer biomarker” is one which indicates the propensity, presence, prognosis, diagnosis, stratification, trend or relation toward a relationship or correlation to a symptom or sign or the etiology of prostate cancer.

An “affiliated molecular partner” is any protein or peptide that is associated with, either by direct binding or by virtue of a functional connection (in the same signaling pathway or disease indication), to a biomarker.

In one aspect of the invention, the prostate cancer biomarkers and affiliated molecular partners include, but are not limited to FASN, USP2a, NPY, AMACR and pAKT. In another aspect of the invention, the presence and/or the concentration of FASN, USP2a, NPY, AMACR or pAKT, or in combination of any two or more proteins, in a sample from a subject, is determined using MRM-MS based assays and methods.

In one aspect of the invention, the concentration of a prostate cancer biomarker and/or affiliated molecular partner is determined by comparing signal of said biomarker and/or said affiliated molecular partner in a sample from a subject with the standard curve created with the peptides and peptides signatures for said biomarker and/or said affiliated molecular partner.

Certain peptides and peptide signatures have been identified which are useful in the determination of the concentration or presence of prostate cancer biomarkers or their affiliated molecular partners. These peptides comprise SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43 and combinations thereof.

In one aspect of the present invention are provided kits for quantifying the level of one or more prostate cancer biomarkers and/or affiliated molecular partners in a sample comprising two, three, four, five or more peptides selected from the peptides identified in the present invention, as listed above.

In another aspect of the present invention are provided the composition of a synthetic peptide with 5, 6, 7, 8, 9, 10, 11, 12, 13, 14, 15, 16 or 17 contiguous amino acids of a peptide selected from any peptides identified in the present invention, as listed above. In addition, the synthetic peptide has a detectable label.

### DETAILED DESCRIPTION OF THE INVENTION

The present invention relates to protein signature assays which utilize multiple reaction monitoring mass spectroscopy (MRM-MS) in the fields of cancer diagnostics and therapeutics. The present invention also provides kits and peptides useful in the methods and assays of the present invention. These assays are useful in research and for the detection, diagnosis, prognosis and treatment of certain types of cancer or lipogenic related pathological conditions, in particular, cancers such as prostate cancers and metabolic syndromes having etiologies implicating FASN, USP2a, NPY, AMACR or pAKT, alone or in combination, or their associated proteins or genes.

Liquid chromatography-multiple reaction monitoring (LC-MS/MRM) coupled with stable isotope labeled dilution of peptide standards has been shown to be an effective method for biomarker verification and validation of novel protein biomarker candidates. Unlike untargeted mass spectrometry frequently used in biomarker discovery studies, targeted MS methods are peptide sequence-based modes of MS that focus the full analytical capacity of the instrument on tens to hundreds of selected peptides in a complex mixture. By restricting detection and fragmentation to only those peptides derived from proteins of interest, sensitivity and reproducibility are improved dramatically compared to discovery-mode MS methods. This method of mass spectrometry-based multiple reaction monitoring (MRM) quantitation of proteins can dramatically impact the discovery and quantitation of biomarkers via rapid, targeted, multiplexed protein expression profiling of clinical samples.

The principle of the multiplex biomarker assay platform MRM-MS allows for target protein sequence to be selected for assay development and measurement with the capability to quantify multiple protein biomarkers in clinical samples in a single assay cycle.

Specifically, MRM-MS allows quantification of a large set of proteins in complex biological samples with high accuracy, by the addition of isotopically labeled peptides or proteins, as internal standards.

The quantification is based on the relative intensity of the analyte signal, compared to the signal of known levels of internal standards.

As used herein, the term “internal standard” refers to one or more proteins or signature peptides for one or more proteins that are added in a constant amount to a biological sample from a subject in a MRM-MS assay. The peptides are then used for calibration by plotting the ratio of the analyte signal to the internal standard signal as a function of analyte concentration of the standards. The peptides used as internal standards are signature peptides for a protein to be analyzed, such as a prostate cancer biomarker and/or affiliated molecular partner.

Disclosed herein is the analysis of the recombinant full length of FASN (Fatty Acid Synthase; GenBank NM_{—}004104; SEQ ID NO. 1, which is encoded by CDS 118-7653 of FASN cDNA, SEQ ID NO. 44), USP2a (ubiquitin specific peptidase 2; GenBank NM_{—}004205 (isoform 1:long variant; SEQ ID NO. 2, which is encoded by CDS 296-2113 of USP2a cDNA, SEQ ID NO. 45), NPY (Neuropeptide Y; GenBank NM_{—}000905; SEQ ID NO. 3, which is encoded by CDS 89-382 of NPY cDNA, SEQ ID NO.46) and AMACR (alpha-methylacyl-CoA racemase, nuclear gene encoding mitochondrial protein, transcript variant 1, OR AMACR IA; GenBank NM_{—}014324; SEQ ID NO. 4, which is encoded by CDS 97-1245 of AMACR IA cDNA, SEQ ID NO. 47) and/or pAKT (v-akt murine thymoma viral oncogene homolog 1; GenBank NM_{—}005163 variant 1: long version; SEQ ID NO. 5, which is encoded by CDS 555-1997 of pAKT cDNA, SEQ ID NO. 48) proteins. The proteins are identified by their signature peptides as internal standards for the mass spectrometry analysis of these proteins.

According to the present invention, the signature peptides for FASN were identified with MRM-MS based assay platform, comprising SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25 and SEQ ID NO. 26.

According to the present invention, the signature peptides for USP2a were identified with MRM-MS based assay platform, comprising SEQ ID NO. 27, SEQ ID NO. 28, SEQ ID NO.29, SEQ ID NO. 30 and SEQ ID NO. 31.

According to the present invention, the signature peptides for NPY were identified with MRM-MS based assay platform, comprising SEQ ID NO. 32 and SEQ ID NO. 33.

According to present invention, the signature peptides for AMACR were identified with MRM-MS based assay platform, comprising SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42 and SEQ ID NO. 43.

According to the present invention, the concentration of an isoform of FASN, USP2a, NPY, AMACR and/or pAKT, contained in a sample obtained from a subject may be determined by treating the sample from the subject to digest the one or more isoforms contained in the sample. After digestion the sample may be analyzed by mass spectrometry to generate a mass spectrometry profile. The mass spectrometry profile of the digested sample may then be compared to a standard curve to calculate the concentration contained in the sample. Liquid chromatography may also be used in the method of analyzing the sample prior to generating the mass spectrometry profile.

As used herein, the “mass spectrometry profile” refers to one or more proteins or a group of peptides from a sample isolated from a subject wherein the presence and the concentration of proteins or peptides, taken individually or together, is indicative/predictive of a certain condition, in particular, a cancer such as prostate cancer and metabolic syndromes.

In some instances, the concentration of FASN determined in the sample obtained from a subject may be selected from one or more isoforms of FASN. The isoforms of FASN may be selected from SEQ ID NO. 1 or variants thereof.

In some instances, the concentration of USP2a determined in the sample obtained from a subject may be selected from one or more isoforms of USP2a. The isoforms of USP2a may be selected from SEQ ID NO. 2 or variants thereof.

In some instances, the concentration of NPY determined in the sample obtained from a subject may be selected from one or more isoforms of NPY. The isoforms of NPY may be selected from SEQ ID NO. 3 or variants thereof.

In some instances, the concentration of AMACR determined in the sample obtained from a subject may be selected from one or more isoforms of AMACR. The isoforms of AMACR may be selected from SEQ ID NO. 4 or variants thereof.

In some instances, the concentration of pAKT determined in the sample obtained from a subject may be selected from one or more isoforms of pAKT. The isoforms of pAKT may be selected from SEQ ID NO. 5 or variants thereof.

In some instances, the concentration of any two, three, four or five proteins selected from the group consisting of FASN, USP2a, NPY, AMACR and pAKT, or isoforms thereof, or variants thereof, is determined in a sample obtained from a subject.

In the methods of the present invention, a sample may be obtained from a subject. While the sample may include any sample which is amendable for protein analysis, it is most often a biofluid sample, more preferably a serum sample. The sample may be obtained from a subject. As used herein, a “subject” refers to a vertebrate, preferably a mammal, more preferably a primate and still more preferably a human.

The sample may be obtained from a subject who is a patient. As used herein, “patient” refers to a subject who may seek or be in need of treatment, requires treatment, is receiving treatment, will receive treatment, or a subject who is under care by a trained professional for a particular disease or condition.

The term “treatment” as used herein, means anything which has the effect of ameliorating, reversing, alleviating, inhibiting the progress of, or preventing, either partially or completely, the growth of tumors, tumor metastases, or other FASN, USP2a, NPY, AMACR or pAKT related pathological conditions. The term “treating” as used herein, unless otherwise indicated, refers to the act of administering treatment.

As used herein, the term “sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid, sputum and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecules.

In one aspect of the invention, the methods are provided with a standard curve to calculate and determine the concentration of one or more prostate biomarkers and/or affiliated molecular partners in a sample from a subject. A calibration standard is selected from the group of peptides consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO.29, SEQ ID NO. 30, SEQ ID NO. 31, SEQ ID NO. 32, SEQ ID NO. 33, SEQ ID NO.34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42 and SEQ ID NO. 43 or combination thereof.

In a further aspect, a series of peptides across a range of concentrations from 0.5 ng/mL to 40 ng/mL are prepared. The signal of a set of peptides with known concentration is measured with MRM-MS assay. A standard calibration curve is created by plotting the changes of the analytic signal with the known concentration of peptides. In a further aspect of the invention, the standard calibration curve is generated with at least three data points within the range of about 1 ng/mL to about 30 ng/mL peptide concentration. In addition, the standard calibration curve has a lower data point at about 0.5 to 1.5 ng/mL peptide concentration, and an upper data point at about 25-35 ng/mL peptide concentration.

The various aspects and embodiments taught herein may further rely on comparing the quantity of any one or more peptides or peptide signatures measured in samples with reference values of the quantity of said one or more peptides or peptide signatures, wherein said reference values represent known predictions, diagnoses and/or prognoses of diseases or conditions as taught herein.

For example, distinct reference values may represent the prediction of a risk (e.g., an abnormally elevated risk) of having a given disease or condition as compared to the prediction of no or normal risk of having said disease or condition. In another example, distinct reference values may represent predictions of differing degrees of risk of having such disease or condition.

In a further example, distinct reference values can represent the diagnosis of a given disease or condition as taught herein as compared to the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein as compared to a poor prognosis for said disease or condition. In a further example, distinct reference values may represent varyingly favorable or unfavorable prognoses for such disease or condition. Such comparison may generally include any means to determine the presence or absence of at least one difference and optionally of the size of such different between values or profiles being compared. A comparison may include a visual inspection, or an arithmetical or statistical comparison of measurements.

As used herein, the term “condition” refers to the status of any cell, tissue, organ, organ system or organism. Conditions may reflect a disease state or simply the physiologic situation of an entity. Conditions may be benign or malignant. In one aspect of the invention, the disease is a cancer or a metabolic syndrome with etiology involved in FASN, USP2a, NPY, AMACR and/or pAKT protein or encoding gene thereof. In a further aspect of the invention, the cancer is a prostate cancer. The term “prostate cancer” means a cancer of the prostate tissue. Reference values for the quantity of any one or more peptides or peptide signatures may be established according to known procedures previously employed for other peptides or peptide signatures.

According to the present invention, the sample, once obtained from the subject, may be subjected to enzyme digestion. As used herein, the term “digest” means to break apart into shorter peptides. As used herein, the phrase “treating a sample to digest proteins” means manipulating a sample in such a way as to break down proteins in a sample. In the present invention, one or more isoforms of FASN or USP2a or NPY or pAKT or AMACR proteins may be digested using enzymes. These enzymes include, but are not limited to, trypsin, endoproteinase Glu-C and chymotrypsin.

The sample may also be spiked with a known concentration of one or more peptides or proteins. As used herein, the term “spike or spiking” refers to the addition of a known compound. The peptides or proteins used to spike the sample may be selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25. SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36 SEQ ID NO. 37, SEQ ID NO. 38, SEQ ID NO. 39, SEQ ID NO. 40, SEQ ID NO. 41, SEQ ID NO. 42, SEQ ID NO. 43 and combinations thereof.

Specifically, the one or more peptides or proteins used to spike the sample isolated from a subject are further labeled with a detectable agent, including, but not limited to a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, etc) and deuterium.

According to the present invention, a synthetic peptide 6-17 amino acids in length is provided. The synthetic peptide, in particular, has at least 5 contiguous amino acids of a peptide selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO.29, SEQ ID NO. 30, SEQ ID NO. 31, SEQ ID NO. 32, SEQ ID NO. 33, SEQ ID NO.34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42 and SEQ ID NO. 43. In addition, the synthetic peptide is 5-18 amino acids in length, 8-17 amino acids in length or 5-15 amino acids in length. More specifically, the synthetic peptide is 5, 6, 7, 8, 9, 11, 12, 13, 14, 15, 16, 17, 18 amino acids in length.

In one aspect of the invention, a synthetic peptide is incorporated with a detectable agent. The detectable agents include, but are not limited to a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, etc) and deuterium.

According to the present invention, a synthetic peptide further comprises a naturally modified amino acid of any peptide. The natural modifications include, but are not limited to, deamination of glutamine and asparagine, amination, oxidation and hydroxylation, etc.

The identified peptides for prostate cancer biomarkers and affiliated molecular partners used in the methods of the present invention are suited for preparation of kits produced in accordance with well-known procedures in the art. The present invention thus provides kits comprising two or more calibration standards, which are used to quantify the concentration of one or more prostate cancer biomarkers or affiliated molecular partners in a sample from a subject. In one aspect of the invention, the kits contain two or more calibration standards selected from the group of peptides consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO.29, SEQ ID NO. 30, SEQ ID NO. 31, SEQ ID NO. 32, SEQ ID NO. 33, SEQ ID NO.34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42 and SEQ ID NO. 43.

Specifically, the kits may contain two or more calibration standards that are synthetic peptides 6 to 17 amino acids in length with at least 5 contiguous amino acids of a peptide selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO.29, SEQ ID NO. 30, SEQ ID NO. 31, SEQ ID NO. 32, SEQ ID NO. 33, SEQ ID NO.34, SEQ ID NO. 35, SEQ ID NO. 36, SEQ ID NO. 37, SEQ ID NO. 38, SEQ ID NO.39, SEQ ID NO. 40, SEQ ID NO. 41, SEQ ID NO 42 and SEQ ID NO. 43.

In another aspect of the invention, the kits comprise two or more calibration standard peptides that are further labeled with a detectable reagent, including, but not limited to a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, etc) and deuterium.

In addition, the kits may contain two or more the calibration standards in at least three different concentrations within the range from 0.5 ng/m L to 35 ng/mL.

The kits may optionally comprise reagents with identifying description or label or instructions relating to their use in the methods of the present invention. In addition, the kits may comprise one or more enzymes to digest proteins in a sample from a subject. The enzymes include, but are not limited to, trypsin, endoproteinase Glu-C and chymotrypsin.

The invention is further illustrated by the following non-limiting examples.

### EXAMPLES

### Example 1

### Generation of Tryptic Peptides

Full length recombinant FASN, USP2a, NPY, AMACR and pAKT protein were purchased from Origene Technologies Inc (Rockville, Md.) and Genway Biotech Inc (San Diego, Calif.). Upon separation on SDS-PAGE by molecular weight, the protein band was excised and subjected to reduction/alkylation followed by trypsin digestion to yield tryptic peptides.

### Example 2

### Selection of Proteotypic Peptides

To select the signature peptides for FASN, USP2a, NPY, AMACR and pAKT including MS2 profiles for each peptide, the Skyline software package (University of Washington, skyline.gs.washington.edu) was used to analyze the experimentally derived data from the in-gel digestion of recombinant protein. Furthermore, the Skyline software can generate a list of SRM transition candidates for each peptide consisting of only y and b ions.

### Example 3

### MRM Method Generation and Optimization

An Agilent 6520 Q-TOF was used to create tryptic peptide profiles for each in-gel digested recombinant protein (Agilent Technologies, Santa Clara, Calif.). The tryptic peptides were then identified using Spectrum Mill and MASCOT, two search engines developed by Agilent and Matrix Science, respectively. MRM methods were generated by Skyline based on the results from MASCOT. Other peptides that were not picked up by Skyline but have high scores (score>6 for Spectrum Mill, score>10 for MASCOT) and good MS2 spectra were manually selected to generate MRM methods.

Up to five transitions were created for each identified peptide. The collision energy (CE) voltage was identical as the CE used in Q-TOF data acquisition. The QQQ is set to operate in a targeted fashion whereby only molecular ions corresponding to the most dominant charge state of +2, +3 or +4 of selected peptides are transmitted through Q1, and MRM transition candidates are monitored in Q3.

Both Q1 and Q3 resolution were set to “unit”, and a default dwell time of 5 or 10 milliseconds was used. At least 2 replicates were performed for each MRM method.

### Example 4

### LC-QQQ-MRM Mass Spectrometry

Liquid chromatography was performed using a 1200 Series LC system interfaced to a 6410 (Nuclea Biotechnologies) Triple Quadrupole (QQQ) LC/MS/MS (Agilent Technologies, Santa Clara, Calif.). Agilent MassHunter software (version B.03.01) was used for data acquisition and processing. The LC separation of peptides was carried out on Zorbax 300SB-C18 5-μm column (Agilent).

For analysis of tryptic peptides, processed peptides were loaded onto the column using an Agilent 1260 autosampler. The gradient separation is performed by the capillary LC pump delivering a mixture of 99.9% water/0.1% formic acid (mobile phase A) and 99.9% acetonitrile/0.1% formic acid (mobile phase B) at 400 μL/min. Peptides were separated at a flow rate of 400 μL/min by a nanopump delivering a linear gradient of 2 to 33.5% mobile phase B in 30 minutes followed by 33.5 to 95% mobile phase B in 1 minute.

The analyses were performed in the positive ionization mode with a capillary voltage set at 4000 V and an electron multiplier voltage (Delta EMV) at 200 V. The drying gas flow rate was 10 L nitrogen/min with an interface heater temperature of 350° C. The MS fragmentor voltage was fixed at 135 V. Multiple reaction monitoring (MRM) transition dwell times are 5 or 10 ms, with both Q1 and Q3 set to “unit”.

### Example 5

### Selected MRM Peptides of FASN and USP2a

The resulting peptide signatures of both FASN and USP2a are given in Tables 1-2. According to the tables, the peptide # represents the MRM peptide number, m/z represents the mass over charge of precursor ion, Sequence represents the peptide sequence of MRM, RT represents the retention time of MRM peptides and MS2 represents the product ions post fragmentation with ranking of peak intensity from 1 to 5.

TABLE 1
Sequence of MRM peptides and MS2 data for FASN

| FASN | | | | | | | | | |
|---|---|---|---|---|---|---|---|---|---|
| Peptide | | SEQ ID | | RT | MS2 | MS | MS | MS | MS |
| Number | Sequence | NO | m/z | (minutes) | Rank1 | Rank2 | Rank3 | Rank4 | Rank5 |
| 1 | AAEQYTPK | 6 | 454.2 | 8.2 | 765.4 | 508.3 | 636.3 | 836.4 | — |
| 2 | VFTTVGSAEK | 7 | 519.8 | 12.1 | 792.4 | 247.1 | 491.2 | 590.3 | 691.4 |
| 3 | AFDTAGnGYCR | 8 | 616.8 | 12.2 | 727.3 | 219.1 | 506.2 | 899.4 | 1014.4 |
| | (n = deaminated | | | | | | | | |
| | Asn) | | | | | | | | |
| 4 | ADEASELACPTPK | 9 | 694.8 | 11.6 | 602.3 | 187.0 | 786.4 | — | — |
| 5 | GYAVLGGER | 10 | 461.2 | 13.5 | 418.2 | 221.1 | 292.1 | 531.3 | 701.4 |
| 6 | YSGTLNLDR | 11 | 519.8 | 14.0 | 788.4 | 517.3 | 630.4 | 731.4 | 875.5 |
| 7 | QELSFAAR | 12 | 461.2 | 12.3 | 317.2 | 551.3 | 664.4 | 246.2 | 258.1 |
| 8 | LQVVDQPLPVR | 13 | 632.4 | 18.6 | 242.1 | 341.2 | 581.4 | 923.5 | 1022.6 |
| 9 | FDASFFGVHPK | 14 | 417.9 | 20.0 | 537.3 | 244.2 | 263.1 | 381.2 | 684.4 |
| 10 | GTPLISPLIK | 15 | 519.8 | 20.7 | 670.4 | 147.1 | 159.1 | 557.4 | 880.6 |
| 11 | SEGVVAVLLTK | 16 | 558.3 | 21.4 | 217.1 | 274.1 | 474.3 | 743.5 | 899.6 |
| 12 | VLEALLPLK | 17 | 498.3 | 22.9 | 783.5 | 213.2 | 357.2 | 470.3 | 654.5 |
| 13 | VVVQVLAEEPEAVLK | 18 | 812.0 | 23.4 | 199.1 | 260.2 | 656.4 | 985.5 | 1098.6 |
| 14 | AFEVSEnGNLVVSGK | 19 | 775.9 | 18.2 | 219.1 | 390.2 | 291.2 | 348.2 | 1104.6 |
| | (n = deamidated | | | | | | | | |
| | Asn) | | | | | | | | |
| 15 | SLLVNPEGPTLmR | 20 | 721.9 | 19.1 | 314.2 | 201.1 | 413.3 | 916.5 | 1030.5 |
| | (m = oxidized | | | | | | | | |
| | Met) | | | | | | | | |
| 16 | GVDLVLNSLAEEK | 21 | 693.9 | 22.0 | 272.1 | 157.1 | 385.2 | 484.3 | 903.5 |
| 17 | SFYGSTFLLCR | 22 | 675.8 | 22.9 | 953.5 | 335.1 | 448.2 | 595.3 | 1116.6 |
| 18 | AALQEELQLCK | 23 | 651.8 | 17.8 | 919.5 | 147.1 | 256.2 | 420.2 | 790.4 |
| 19 | VGDPQELnGITR | 24 | 650.3 | 14.2 | 514.8 | 561.3 | 572.3 | 674.4 | 803.4 |
| | (n = deaminated | | | | | | | | |
| | Asn) | | | | | | | | |
| 20 | VYQWDDPDPR | 25 | 645.8 | 15.8 | 272.2 | 484.3 | 599.3 | 714.3 | 900.4 |
| 21 | TGTVSLEVR | 26 | 481.3 | 13.8 | 603.3 | 159.1 | 260.1 | 274.2 | 702.4 |

TABLE 2
Sequence of MRM peptides and MS2 data for USP2a

| USP2a | | | | | | | | | |
|---|---|---|---|---|---|---|---|---|---|
| Peptide | | | | RT | MS | MS | MS | MS | MS |
| Number | Sequence | SEQ ID NO | m/z | (minutes) | Rankl | Rank2 | Rank3 | Rank4 | Rank5 |
| 1 | LDSQSDLAR | 27 | 502.8 | 8.4 | 229.1 | 776.4 | 561.3 | 891.4 | 359.2 |
| 2 | FLLDGLHNEVNR | 28 | 476.3 | 17.4 | 261.2 | 631.3 | 768.4 | 388.2 | 517.3 |
| 3 | TYGPSSLLDYDR | 29 | 693.8 | 18.2 | 1122.5 | 265.1 | 681.3 | 568.2 | 322.1 |
| 4 | IGDLFVGQLK | 30 | 545.3 | 19.1 | 691.4 | 286.1 | 544.3 | 976.5 | 260.2 |
| 5 | LTTFVNFPLR | 31 | 604.3 | 21.7 | 646.4 | 745.4 | 385.3 | 892.5 | — |

From the data it is evident that the MRM assay utilizing the Agilent QQQ 6410 mass spectrometer yielded optimal sequence coverage for both FASN and USP2a. These signature peptides will be useful, either alone or in combination, as standards for the quantitation of the proteins in biological samples, tissues, organs or whole organisms.

### Example 6

### Selected MRM Peptides of Neuropeptide Y (NPY)

Following the experimental design of Examples 1-4 the resulting peptide signatures of NPY (Neuropeptide Y; GenBank NM_{—}000905; SEQ ID NO: 3) are given in Tables 3. According to the tables, the peptide # represents the MRM peptide number, represents the mass over charge of precursor ion, Sequence represents the peptide sequence of MRM, RT represents the retention time of MRM peptide, and MS2 represents the product ions post fragmentation with ranking of peak intensity from 1 to 5.

TABLE 3
Sequence of MRM peptides and MS2 data for NPY

| NPY | | | | | | | | | |
|---|---|---|---|---|---|---|---|---|---|
| Peptide | | | | RT | MS | MS | MS | MS | MS |
| Number | Sequence | SEQ ID NO | m/z | (minutes) | Rankl | Rank2 | Rank3 | Rank4 | Rank5 |
| 1 | YYSALR | 32 | 386.7 | 9.4 | 446.3 | 327.1 | 609.3 | 175.1 | — |
| 2 | ESTENVPR | 33 | 466.2 | 6.2 | 272.2 | 485.3 | 371.2 | 614.3 | 217.1 |

### Example 7

### Selected MRM Peptides of Alpha-Methylacyl-CoA Racemase (AMACR)

Following the experimental design of Examples 1-4 the resulting peptide signatures of AMACR (alpha-methylacyl-CoA racemase, nuclear gene encoding mitochondrial protein, transcript variant 1, or AMACR IA; GenBank NM_{—}014324; SEQ ID NO. 4) are given in Tables 4. According to the tables, the peptide # represents the MRM peptide number, m/z represents the mass over charge of precursor ion, Sequence represents the peptide sequence of MRM, RT represents the retention time of MRM peptide and MS2 represents the product ions post fragmentation with ranking of peak intensity from 1 to 5.

TABLE 4
Sequence of MRM peptides and MS2 data for AMACR

| AMACR | | | | | | | | | |
|---|---|---|---|---|---|---|---|---|---|
| Peptide | | | | RT | MS | MS | MS | MS | MS |
| Number | Sequence | SEQ ID NO | m/z | (minutes) | Rankl | Rank2 | Rank3 | Rank4 | Rank5 |
| 1 | EEIYQLNSDK | 34 | 619.8 | 12.9 | 259.1 | 576.3 | 867.4 | 463.2 | 704.4 |
| 2 | SSLWEAPR | 35 | 473.2 | 14.3 | 658.3 | 343.2 | 386.2 | 771.4 | — |
| 3 | LSGFGQSGSFCR | 36 | 651.8 | 14.7 | 713.3 | 898.4 | 626.3 | — | — |
| 4 | LSGFGQSGSFCR | 36 | 434.9 | 14.7 | 626.3 | 713.3 | 482.2 | 569.3 | 405.2 |
| 5 | FADVFAK | 37 | 399.2 | 15.0 | 579.3 | 650.3 | 464.3 | — | — |
| 6 | EEIYQLNSDKIIESNK | 38 | 641.7 | 16.9 | 832.9 | 776.4 | 694.9 | — | — |
| 7 | SLVLDLK | 39 | 394.2 | 17.2 | 587.4 | 488.3 | 260.2 | 201.1 | — |
| 8 | RSDVLLEPFR | 40 | 411.2 | 17.5 | 419.2 | 571.3 | 458.2 | 661.4 | 684.4 |
| 9 | LQLGPEILQR | 41 | 583.8 | 19.7 | 242.1 | 925.5 | 812.5 | 755.4 | 355.2 |
| 10 | LQLGPEILQR | 41 | 389.6 | 19.7 | 416.3 | 303.2 | 529.3 | 638.4 | 242.1 |
| 11 | SDVLLEPFR | 42 | 538.3 | 20.0 | 302.1 | 661.4 | 548.3 | 419.2 | 528.3 |
| 12 | LAGHDINYLALSGVLSK | 43 | 591.0 | 22.2 | 774.5 | 703.4 | 590.4 | 884.4 | 503.3 |

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

1. A method of determining the concentration of a prostate cancer biomarker or affiliated molecular partner in a sample comprising:
(a) obtaining a sample from a subject;
(b) treating said sample to digest one or more prostate cancer biomarkers or affiliated molecular partners contained therein;
(c) generating a mass spectrometry profile of the digested sample of step (b);
(d) comparing the mass spectrometry profile from step (c) to a standard curve, wherein said standard curve has been created using at least one calibration standard selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO 28, SEQ ID NO. 29 SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43 and combinations thereof; and
(e) calculating a concentration of said one or more prostate cancer biomarkers or affiliated molecular partners in the sample obtained from the subject based on the standard curve.

2. The method of claim 1, wherein the prostate cancer biomarker is FASN.

3. The method of claim 1, wherein the affiliated molecular partner is USP2a.

4. The method of claim 2, wherein the affiliated molecular partner is USP2a.

5. The method of claim 2 wherein the sample of step (b) is subjected to liquid chromatography prior to the generation of said mass spectrometry profile.

6. The method of claim 1, further comprising spiking the sample of (a) with a known concentration of one or more peptides or proteins selected from the group consisting of SEQ ID NO. 1, SEQ ID NO. 2, SEQ ID NO. 3, SEQ ID NO. 4, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO 28, SEQ ID NO. 29 SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43 and combinations thereof.

7. The method of claim 6, wherein said one or more peptides or proteins comprises a detectable label.

8. The method of claim 7, wherein the detectable label is selected from the group consisting of a fluorescent label, nitrogen-15, carbon-13 and deuterium.

9. The method of claim 1, wherein said prostate cancer biomarker is FASN and comprises SEQ ID NO. 1.

10. The method of claim 1, wherein said affiliated molecular partner is USP2a and comprises SEQ ID NO. 2.

11. The method of claim 1, wherein the subject is a patient.

12. The method of claim 1, wherein the sample is serum.

13. The method of claim 12, wherein the sample obtained from the subject is treated to deplete at least one serum protein contained therein before digestion.

14. The method of claim 1, wherein the sample is obtained from said subject prior to administration to said subject of any treatment for cancer.

15. The method of claim 1, wherein the standard curve is generated having a lower data point at about 0.5 to 1.5 ng/mL protein concentration, an upper data point at 25 to 35 ng/mL protein concentration.

16. The method of claim 15, wherein the standard curve is generated using at least 3 data points within the range of about 1 to about 30 ng/mL protein concentration.

17. The method of claim 15, wherein the standard curve is generated having a lower data point at about 0.5 to 1.5 ng/mL protein concentration, an upper data point at 25 to 35 ng/mL peptide concentration, and at least 3 data points in the range of about 1 ng/mL to about 30 ng/mL peptide concentration.

18. The method of claim 1, wherein the standard curve has a maximum bias of no more than 20%.

19. The method of claim 1, where digestion is performed using trypsin.

20. The method of claim 1, where digestion is performed using endoproteinase Glu-C.

21. The method of claim 1, where digestion is performed using chymotrypsin.

22. The method of claim 1, wherein the mass spectrometry is MRM mass spectrometry with QQQ.

23. A kit used to quantify the level of one or more prostate cancer biomarkers or affiliated molecular partners in a sample comprising two or more calibration standards selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43 and combinations thereof.

24. A synthetic isolated peptide 6 to 17 amino acids in length having at least 5 contiguous amino acids of a peptide selected from the group consisting of SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO 28, SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43.

25. The peptide of claim 23 where the peptide is from 6 to 17 amino acids in length.

26. The peptide of claim 24 further comprising at least one detectable label.

27. The peptide of claim 25 wherein the at least one detectable label is selected from the group consisting of a fluorescent label, nitrogen-15, carbon-13 and deuterium.

28. The method of claim 1, wherein the prostate cancer biomarker is NPY.

29. The method of claim 2, wherein the affiliated molecular partner is NPY.

30. The method of claim 1, wherein said prostate cancer biomarker is NPY and comprises SEQ ID NO. 3.

31. The method of claim 1, wherein said affiliated molecular partner is NPY and comprises SEQ ID NO. 3.

32. The method of claim 1, wherein said affiliated molecular partner is AMACR.

33. The Method of claim 2, wherein said affiliated molecular partner is AMACR.

34. The method of claim 2, wherein said affiliated molecular partner is AMACR and comprises SEQ ID NO. 4.

35. The method of claim 28, wherein said affiliated molecular partner is AMACR and comprises SEQ ID NO. 4.
